(19) United States
(12) Patent Application Publication
(10) Pub. No.: US 2009/0118239 A1
(43) Pub. Date: May 7, 2009

(54) Amorphous and crystalline forms of ibandronate disodium

(75) Inventors: Sharon Avhar-Maydan, Givataym (IL); Claude Singer, Kfar Saba (IL); Tamas Koltai, Netanya (IL)

Correspondence Address: KENYON & KENYON LLP, ONE BROADWAY, NEW YORK, NY, 10004, US

(73) Assignee: Teva Pharmaceutical Industries Ltd.

(21) Appl. No.: 12/152,808

(22) Filed: May 15, 2008

Related U.S. Application Data
(60) Provisional application No. 61/001,974, filed on Nov. 5, 2007.

(51) Int. Cl. A61K 31/663; C07F 9/38; A61P 19/10

(52) U.S. Cl. 514/114; 564/15

(57) ABSTRACT

Provided are amorphous and crystalline forms of ibandronate disodium, as well as processes for the preparation thereof.

### CROSS-REFERENCE TO RELATED APPLICATIONS

The present application claims the benefit U.S. Provisional Patent Application No. 61/001974 filed Nov. 5, 2007. The contents of which is incorporated herein by reference.

### FIELD OF THE INVENTION

The invention encompasses amorphous and crystalline forms of ibandronate disodium, as well as processes for the preparation thereof.

### BACKGROUND OF THE INVENTION

Ibandronate sodium, (1-hydroxy-3-(N-methyl-N-pentylamino)propylidene) bisphosphonic acid monosodium salt, is a third-generation nitrogen-containing bisphosphonate characterized by an aliphatic tertiary amine side chain. lbandronate sodium is typically a white powder. Ibandronate sodium has the empirical formula C₉H₂₂NO₇P₂Na and the following chemical structure.

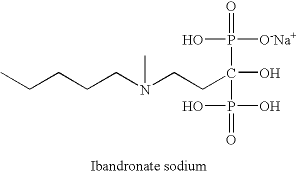

Ibandronate sodium is currently marketed in the United States by Hoffmann-La Roche under the tradename BONIVA® in its monohydrate form. BONIVA® is indicated for the treatment and prevention of osteoporosis in post-menopausal women. BONIVA® is available as an intravenous injection administered every 2-3 months or as an oral formulation. BONIVA® is marketed in Europe under the tradename BONDRONAT® for the treatment of skeletal-related events in patients with breast cancer and bone metastases. BONDRONAT® is available in an ampoule with 1 ml concentrate for solution for infusion; 1 ml of solution is reported to contain 1.125 mg of ibandronic monosodium salt monohydrate, corresponding to 1 mg of ibandronic acid.

Ibandronate salts, such as ibandronate sodium, are generally prepared from ibandronic acid (“IBD-Ac”), which has the following chemical structure:

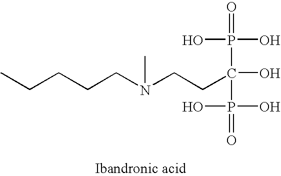

U.S. Pat. No. 4,927,814 discloses diphosphonic acids, such as ibandronic acid, derivatives thereof, processes for preparing the acids and derivatives, and pharmaceutical compositions containing them.

The invention relates to the solid state physical properties of ibandronate sodium. These properties can be influenced by controlling the conditions under which ibandronate sodium is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must necessitate the use of glidants such as colloidal silicon dioxide, talc, starch, or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally administered active ingredient can reach the patient's bloodstream. The rate of dissolution is also a consideration in formulation syrups, elixirs, and other liquid medicaments. The solid state form of a compound can also affect its behavior on compaction and its storage stability.

These practical physical characteristics are influenced by the conformation and orientation of molecules in the unit cell, which define a particular polymorphic form of a substance. The polymorphic form can give rise to thermal behavior different from that of the amorphous material or another polymorphic form. Thermal behavior is measured in the laboratory by such techniques as capillary melting point, thermogravimetric analysis (“TGA”), and differential scanning calorimetry (“DSC”) and can be used to distinguish some polymorphic forms from others. A particular polymorphic form can also give rise to distinct spectroscopic properties that can be detectable by powder x-ray crystallography, solid state ¹³C NMR spectroscopy, and infrared spectrometry.

Generally, a crystalline solid has improved chemical and physical stability over the amorphous form, and forms with low crystallinity. Crystalline forms may also exhibit improved solubility, hygroscopicity, bulk properties, and/or flowability.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

P.C.T. publication No. WO 2006/024024 and U.S. provisional application Ser. No. 60/954,959, filed Aug. 9, 2007, refer to several crystalline forms of ibandronate sodium and processes for their preparation.

There is a need in the art for additional forms of ibandronate sodium.

### SUMMARY OF THE INVENTION

In one embodiment, the invention encompasses ibandronate disodium.

In another embodiment, the invention encompasses solid form of ibandronate disodium.

In another embodiment, the invention encompasses crystalline form of ibandronate disodium.

In another embodiment, the invention encompasses hydrate form of ibandronate disodium.

In one embodiment, the invention encompasses an amorphous form of ibandronate disodium.

In one embodiment, the invention encompasses amorphous ibandronate disodium prepared by a process comprising combining ibandronic acid, water, a base and a source of sodium to obtain a solution, and combining the solution with acetone to precipitate amorphous form.

In another embodiment, the invention encompasses a process for preparing the amorphous ibandronate disodium comprising dissolving ibandronic acid in water; adding a base and a source of sodium ions to the solution; heating the solution; adding acetone to the solution obtain a slurry; and cooling the slurry to precipitate amorphous ibandronate disodium.

In another embodiment, the invention encompasses a crystalline form of ibandronate disodium denominated Form DS2. The crystalline ibandronate disodium Form DS2 is characterized by x-ray powder diffraction reflections at 4.2, 12.8, 17.6, 19.9 and 20.3° 2θ±0.2° 2θ.

In another embodiment, the invention encompasses crystalline ibandronate disodium Form DS2 having a maximal particle size of less than about 500 μm.

In another embodiment, the invention encompasses a process for preparing ibandronate disodium Form DS2 by combining ibandronic acid, water, a base and a source of sodium to obtain a solution, and combining the solution with acetone to obtain a slurry (heterogeneous mixture), and maintaining the slurry to precipitate crystalline ibandronate disodium Form DS2.

In another embodiment, the invention encompasses a process for preparing the crystalline ibandronate disodium Form DS2 comprising dissolving ibandronic acid in water; heating the solution; adding a base and a source of sodium ions to the solution; heating the solution; adding acetone to obtain a slurry; and cooling the slurry to precipitate the crystalline ibandronate disodium Form DS2.

In another embodiment, the invention encompasses a crystalline form of ibandronate disodium denomiated Form DS3. The crystalline ibandronate disodium is characterized by x-ray powder diffraction reflections at 4.6, 9.5, 14.9, and 17.3° 2θ±0.2 ° 2θ.

In another embodiment, the invention encompasses a process for preparing the crystalline ibandronate disodium Form DS3 comprising storing amorphous ibandronate disodium at a temperature of about 10° C. to about 30° C., at about 80% to about 100% relative humidity, for about 2 days to about 7 days to obtain crystalline ibandronate disodium Form DS3.

In another embodiment, the invention encompasses crystalline ibandronate disodium Form DS3 having a maximal particle size of less than about 500 μm.

In another embodiment, the invention encompasses a pharmaceutical formulation comprising a therapeutically effective amount of at least one of the above-described forms of ibandronate disodium, and at least one pharmaceutically acceptable excipient.

In another embodiment, the invention encompasses a process for preparing a pharmaceutical formulation comprising combining at least one of the above-described forms of ibandronate disodium with at least one pharmaceutically acceptable excipient.

In another embodiment, the invention encompasses the use of the above-described forms of ibandronate disodium in the manufacture of a pharmaceutical composition.

In another embodiment, the invention encompasses methods of treating or preventing skeletal-related events, such as osteoporosis, comprising administering a pharmaceutical formulation comprising a therapeutically effective amount of at least one of the above-described forms of ibandronate disodium and at least one pharmaceutically acceptable excipient to a patient in need thereof.

### BRIEF DESCRIPTION OF THE DRAWINGS

FIG. 1 illustrates a characteristic x-ray powder diffractogram of amorphous ibandronate disodium.

FIG. 2 illustrates a characteristic x-ray powder diffractogram of crystalline ibandronate disodium Form DS2.

FIG. 3 illustrates a characteristic x-ray powder diffractogram of crystalline ibandronate disodium Form DS3.

FIG. 4 illustrates a characteristic x-ray powder diffractogram of crystalline ibandronate disodium Form DS4.

### DETAILED DESCRIPTION OF THE INVENTION

The invention addresses a need in the art by providing additional crystalline forms of ibandronate disodium, as well as processes for their preparation. The invention also provides an amorphous form of ibandronate disodium.

As used herein, unless otherwise defined, the term “room temperature” refers to a temperature of about 15° C. to about 30° C.

The invention encompasses ibandronate disodium.

The invention encompasses ibandronate disodium in solid form.

The invention encompasses a crystalline form of ibandronate disodium.

The invention encompasses hydrate form of ibandronate disodium.

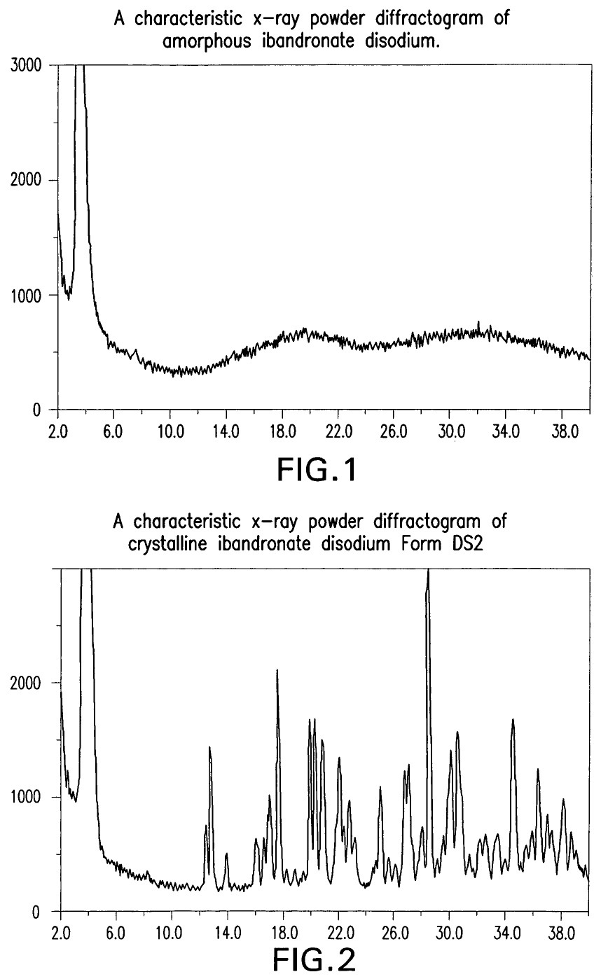

The invention encompasses amorphous form of ibandronate disodium. The amorphous form of ibandronate disodium is characterized by an x-ray powder diffraction (“PXRD”) pattern as depicted in FIG. 1. The amorphous form preferably has less than about 50% crystalline material, more preferably less than about 20%, and most preferably less than about 10%, and most preferably less than 5%, and most preferably less than 1% crystalline material as measured by area percentage XRD.

Amorphous ibandronate disodium can be prepared by combining ibandronic acid, water, a base and a source of sodium to obtain a solution, and combining the solution with acetone to precipitate amorphous form. In one embodiment, the amorphous ibandronate disodium is prepared by a process comprising dissolving ibandronic acid in water; adding a base and a source of sodium ions to the solution; heating the solution; adding acetone to the solution obtain a slurry; and cooling the slurry to precipitate amorphous ibandronate disodium.

Preferably, the ibandronic acid is dissolved in water at a temperature of about 15° C. to about 35° C., preferably, at about room temperature.

The base and the source of sodium ions may be the same or different, and are preferably the same. Preferably, the base and the source of sodium ions is NaOH or Na₂CO₃.

The solution can be heated to a temperature of about 90° C. to about 100° C., preferably to about reflux temperature.

The acetone can be added drop-wise to the solution. The acetone can be added while stirring the solution.

The resulting slurry after addition of acetone can be cooled to a temperature of about 15° C. to about 35° C., preferably, to about room temperature to precipitate the amorphous ibandronate disodium. After cooling, an additional amount of acetone is added to the slurry.

The amorphous ibandronate disodium may be recovered from the slurry by any method known to one of ordinary skill in the art. Preferably, the amorphous ibandronate disodium is recovered by collecting the precipitate from the slurry by filtration. The precipitate can be washed and dried. Preferably, the precipitate is washed with acetone. The precipitate can be dried in a vacuum (pressure of below 100 mmHg) oven at a temperature of about 50° C. to about 80° C., and more preferably at a temperature of about 50° C. The drying can be done under a pressure of about 20 to about 30 mbar. Preferably, the drying is performed until a constant weight is obtained, and more preferably for about 19-20 hours.

The invention also encompasses a crystalline form of ibandronate disodium denominated Form DS2. The crystalline ibandronate disodium Form DS2 is characterized by PXRD reflections at 4.2, 12.8, 17.6, 19.9, and 20.3° 2θ±0.2° 2θ. The crystalline ibandronate disodium Form DS2 can be further characterized by PXRD reflections at 12.4, 13.9, 17.0, 22.0, and 25.0 ±0.2° 2θ.

The crystalline ibandronate disodium Form DS2 may be characterized by PXRD reflections at about 4.2, 12.8 and 17.6° 2θ±0.2° 2θ, and two more peaks selected from the group consisting of: 12.4, 13.9, 16.0, 17.0, 19.9, 20.3, 20.8, 22.0, 22.7, and 25.0 ±0.2° 2θ. Preferably, the crystalline ibandronate disodium Form DS4 is characterized by PXRD reflections at about 4.2, 12.4, 12.8, 16.0 and 17.6° 2θ±0.2° 2θ.

The crystalline ibandronate disodium Form DS2 may be characterized by PXRD reflections at about 12.4, 12.8 and 17.6° 2θ±0.2° 2θ, and two more peaks selected from the group consisting of: 4.2, 13.9, 16.0, 17.0, 19.9, 20.3, and 20.8 ±0.2° 2θ. Preferably, the crystalline ibandronate disodium Form DS2 is characterized by PXRD reflections at about 12.4, 12.8, 17.0, 17.6 and 19.9° 2θ±0.2° 2θ.

The crystalline ibandronate disodium Form DS2 can be even further characterized by a PXRD pattern as depicted in FIG. 2. The crystalline ibandronate disodium Form DS2 shows about 9.1% weight loss by TGA when heated from about 25° C. to about 180° C. Water content of about 9.1% was determined by Karl Fisher (KF). According to the TGA and KF results Form DS2 is hydrate form.

One of ordinary skill in the art is aware that there is a certain amount of experimental error inherent in PXRD techniques. See, e.g., U.S. PHARMACOPEIA, 387-89 (30th ed. 2007), hereby incorporated by reference. As to individual peaks, peak positions are reported over a range of ±0.2° 2θ to account for this experimental error. As to PXRD patterns in their entirety, the term “as depicted” in a particular figure is meant to account for this experimental error, as well as for variations in peak position and intensity due to factors such as, for example, variations in sample preparation, instrumentation, and the skill of the operator of the instrument. A PXRD pattern “as depicted” in a particular figure means that one of ordinary skill in the art, understanding the experimental error involved in powder x-ray diffraction techniques, would determine that the PXRD pattern corresponds to the same crystalline structure as the PXRD pattern depicted in the figure. As used throughout, the PXRD has been measured using themethod and instrumentation described in the Examples section.

The crystalline ibandronate disodium Form DS2 can be prepared by combining ibandronic acid, water, a base and a source of sodium to obtain a solution, and combining the solution with acetone to obtain a slurry (heterogeneous mixture), and maintaining the slurry to precipitate crystalline ibandronate disodium Form DS2. In one embodiment, the crystalline ibandronate disodium Form DS2 is prepared by a process comprising dissolving ibandronic acid in water; heating the solution; adding a base and a source of sodium ions to the solution; heating the solution; adding acetone to obtain a slurry; maintaining and cooling the slurry to precipitate the crystalline ibandronate disodium Form DS2. Without being bound by any theory, it is believed that Form DS2 is obtained through amorphous form. When the slurry is maintained for a period of more than about 20 hours, more preferably more than about 40 hours, amorphous form transitions into Form DS2. Preferably the slurry can be maintained at room temperature for a time period of 25 hours or more, more preferably 40 hours or more, particularly 60 hours or more, or 80 hours or more. A ratio of acetone to water of about 3 or more (v/v) may also favor formation of amorphous form.

The ibandronic acid can be dissolved in water at a temperature of about 15° C. to about 35° C., preferably, at about room temperature.

The base and the source of sodium ions may be the same or different, and are preferably the same. Preferably, the base and the source of sodium ions is NaOH or Na₂CO₃.

The solution can be heated to a temperature of about 90° C. to about 100° C., preferably, to about reflux temperature.

The acetone can be added drop-wise to the solution. The acetone can be added while stirring the solution.

The resulting slurry can be cooled to a temperature of about 10° C. to about 35° C., preferably, to about room temperature to precipitate the crystalline ibandronate disodium Form DS2.

After cooling, an additional amount of acetone can be added to the slurry.

The crystalline ibandronate disodium Form DS2 may be recovered from the slurry by any method known to one of ordinary skill in the art. Preferably, the crystalline ibandronate disodium Form DS2 is recovered by collecting the precipitate from the slurry by filtration. The precipitate can be washed and dried. The precipitate is dried in a vacuum (pressure of less than about 100 mmHg) oven at a temperature of about 50° C. to about 80° C., and more preferably at a temperature of about 50° C. The drying can be done under a pressure of about 20 to about 30 mbar. Preferably, the drying is performed until a constant weight is obtained, and more preferably for about 19-20 hours.

The invention also encompasses crystalline ibandronate diNa Form DS2 having a maximal particle size of less than about 500 μm, more preferably less than about 300 μm, even more preferably less than about 200 μm, even more preferably less than about 100 μm, and most preferably less than about 50 μm. As used herein, unless otherwise defined, the term “maximal particle size,” when used to described a sample of crystalline ibandronate disodium, means that 99% of the particles in the sample have a particle size of less than or equal to the maximal particle size. The particle size of the ibandronate disodium crystalline forms may be measured by methods such as sieves, sedimentation, electrozone sensing (coulter counter), microscopy, and/or Low Angle Laser Light Scattering (LALLS). Preferable, Low Angle Laser Light Scattering is used.

In a preferred embodiment in the processes for making amorphous and DS2 that the base and source of sodium ion is preferably the same, and is preferably NaOH. The source of sodium ions is preferably added in an amount of between about 1.9 to about 3.0 equivalents, more preferably about 1.9 to about 2.5 equivalents, most preferably about 1.9 to about 2.1 equivalents, and particularly about 2 equivalents. The ratio of ibandronic acid to water is preferably 50 grams of the acid to about 5 to 100 ml of water, more preferably about 30 ml water. For amorphous form, the ratio of acetone to water may be about 1 to about 10, preferably about 3 to about 10, and most preferably about 3 (v/v). For amorphous form, acetone can be added in two stages both at start and end of the slurry process. For Form DS2, the ratio of acetone to water may be about 0.5 to about 5, preferably about 0.5 to about 2.5, and most preferably about 2 (v/v).

The invention also encompasses a crystalline form of ibandronate disodium denominated Form DS3. The crystalline ibandronate disodium Form DS3 is characterized by PXRD reflections at 4.6, 9.5, 14.9, and 17.3° 2θ±0.2° 2θ. The crystalline ibandronate disodium Form DS3 can be further characterized by PXRD reflections at 9.0, 11.9, 21.5, 23.7, and 27.5° 2θ±0.2° 2θ.

The crystalline ibandronate disodium Form DS3 may be characterized by PXRD reflections at 4.6, 9.5 and 14.9° 2θ±0.2° 2θ, and two more peaks selected from the group consisting of: 9.0, 11.9, 16.0, 16.7, 17.3, 18.1, 21.0, and 22.5±0.2° 2θ. Preferably, the crystalline ibandronate disodium Form DS3 is characterized by PXRD reflections at 4.6, 9.0, 9.5, 14.9 and 17.3° 2θ±0.2° 2θ.

The crystalline ibandronate disodium Form DS3 may be characterized by PXRD reflections at 9.5, 14.9 and 17.3° 2θ±0.2° 2θ, and two more peaks selected from the group consisting of: 9.0, 11.9, 16.0, 16.7, 18.1, 21.0, and 22.5±0.2° 2θ. Preferably, the crystalline ibandronate disodium Form DS3 is characterized by PXRD reflections at 9.0, 9.5, 14.9, 16.0 and 17.3° 2θ±0.2° 2θ.

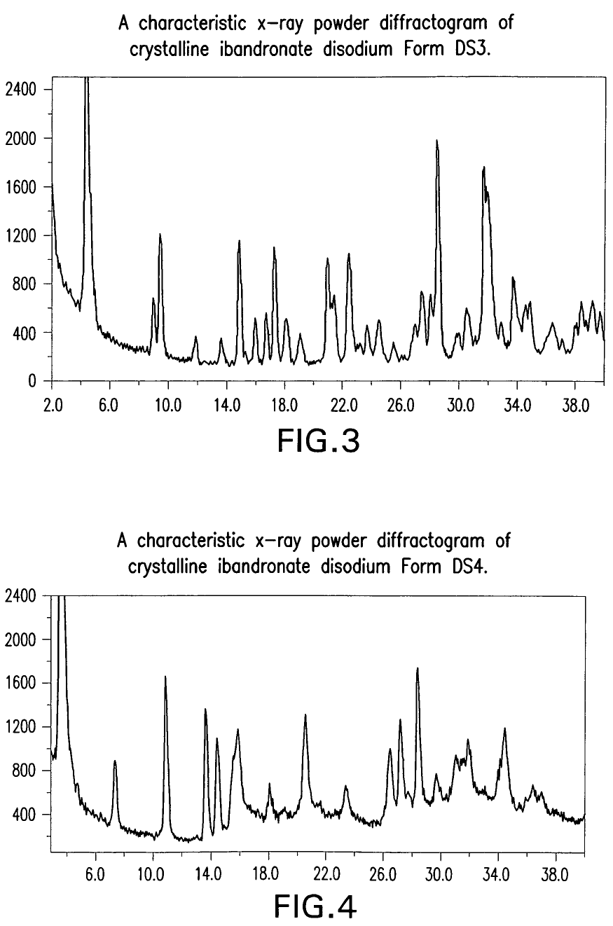

The crystalline ibandronate disodium can be further characterized by a PXRD pattern as depicted in FIG. 3.

The crystalline ibandronate disodium Form DS3 may be prepared by contacting ibandronate disodium with water vapors. In one embodiment, the process comprises storing amorphous ibandronate disodium at a temperature of about 10° C. to about 30° C., at about 80% to about 100% relative humidity, for preferably about 2 days to about 7 days to obtain crystalline ibandronate disodium Form DS3.

The amorphous ibandronate disodium can be stored at about room temperature.

Preferably, the amorphous ibandronate disodium can be stored for about 7 days.

Preferably, the amorphous ibandronate disodium can be stored at about 80% to about 100% relative humidity.

The invention also encompasses crystalline ibandronate disodium Form DS3 having a maximal particle size of less than about 500 μm, more preferably less than about 300 μm, even more preferably less than about 200 μm, even more preferably less than about 100 μm, and most preferably less than about 50 μm.

The invention also encompasses a crystalline form of ibandronate disodium denominated Form DS4. The crystalline ibandronate disodium Form DS4 is characterized by PXRD reflections at 3.7, 10.9 and 14.4° 2θ±0.2° 2θ, and two more peaks selected from the group consisting of: 7.3, 13.6, 15.9, 20.7±0.2° 2θ. Preferably, the crystalline ibandronate disodium Form DS4 is characterized by PXRD reflections at 3.7, 7.3, 10.9, 13.6 and 14.4° 2θ±0.2° 2θ. The invention also encompasses a crystalline form of ibandronate disodium denominated Form DS4.

The crystalline ibandronate disodium Form DS4 may be characterized by PXRD reflections at 10.9, 13.6 and 14.4° 2θ±0.2° 2θ, and two more peaks selected from the group consisting of: 7.3, 13.6, 15.9, 20.7, 22.6, and 27.4±0.2° 2θ. Preferably, the crystalline ibandronate disodium Form DS4 is characterized by PXRD reflections at 7.3, 10.9, 13.6, 14.4 and 15.9° 2θ±0.2° 2θ. The crystalline ibandronate disodium can be even further characterized by a PXRD pattern as depicted in FIG. 4.

The crystalline ibandronate disodium Form DS4 may be prepared by contacting amorphous ibandronate disodium with water vapors, preferably less than about 75%, such as about 40% to about 75%. In one embodiment, the a process comprising comprises storing amorphous ibandronate disodium at a temperature of about 10° C. to about 30° C., at about 60% relative humidity, for preferably about 2 days to about 7 days to obtain crystalline ibandronate disodium Form DS4.

The amorphous ibandronate disodium is can be stored at about room temperature.

Preferably, the amorphous ibandronate disodium can be stored for about 7 days.

The invention also encompasses crystalline ibandronate disodium Form DS4 having a maximal particle size of less than about 500 μm, more preferably less than about 300 μm, even more preferably less than about 200 μm, even more preferably less than about 100 μm, and most preferably less than about 50 μm.

The invention also encompasses a pharmaceutical formulation comprising a therapeutically effective amount of at least one of the above-described crystalline forms of ibandronate disodium, and at least one pharmaceutically acceptable excipient.

The invention further encompasses a process for preparing a pharmaceutical formulation comprising combining at least one of the above-described crystalline forms of ibandronate disodium with at least one pharmaceutically acceptable excipient.

The invention further encompasses the use of the above-described crystalline forms of ibandronate disodium in the manufacture of a pharmaceutical composition.

Pharmaceutical formulations of the invention contain ibandronate disodium, such as one of the above-described forms, and optionally one or more other forms of ibandronate disodium. In addition to the active ingredient, the pharmaceutical formulations of the invention can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. CARBOPOL®), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®), and starch.

Glidants can be added to improve the flowability of a non-compacted solid composition and to improve the accuracy of dosing. Excipients that can function as glidants include colloidal silicon dioxide, mnagnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the invention include maltol, vanillin, ethyl vanillin, menthol, citric acid, fumaric acid, ethyl maltol, and tartaric acid.

Solid compositions can also be dyed using any pharmaceutically acceptable colorant to improve their appearance and/or facilitate patient identification of the product and unit dosage level.

The solid compositions of the invention include powders, granulates, aggregates, and compacted compositions. The dosages include dosages suitable for oral, buccal, rectal, parenteral (including subcutaneous, intramuscular, and intravenous), inhalant, and ophthalmic administration. The dosages can be conveniently presented in unit dosage form and prepared by any of the methods well-known in the pharmaceutical arts.

Solid dosage forms include tablets, powders, capsules, suppositories, sachets, troches, and lozenges, as well as suspensions.

The dosage form of the invention can be a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the invention can comprise any of the aforementioned blends and granulates that were described with reference to tableting, but they are not subjected to a final tableting step.

The invention also encompasses methods of treating or preventing skeletal-related events, such as osteoporosis, comprising administering a pharmaceutical formulation comprising a therapeutically effective amount of at least one of the above-described forms of ibandronate disodium and at least one pharmaceutically acceptable excipient to a patient in need thereof. Ibandronate disodium may be formulated for administration to a mammal, preferably a human, by injection. The crystalline ibandronate disodium can be formulated, for example, as a suspension for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. See, e.g., Ansel, H. C., et al., Pharmaceutical Dosage Forms and Drug Delivery Systems (7th ed. 1999), which is incorporated herein by reference.

BONIVA® and/or BONDRONAT® can be used as guidance for formulation. BONIVA® is available as an intravenous injection administered every 2-3 months and as an oral formulation. BONDRONAT® is available in ampoule with 1 ml concentrate for solution for infusion. 1 ml of the solution contains 1.125 mg of ibandronic monosodium salt monohydrate, corresponding to 1 mg of ibandronic acid.

Having thus described the invention with reference to particular preferred embodiments, those in the art can appreciate modifications to the invention as described and illustrated that do not depart from the spirit and scope of the invention as disclosed in the specification. The following examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Brittain, H. G., Polymorphism in Pharmaceutical Solids, Drugs and the Pharmaceutical Sciences, vol. 95 (Marcel Dekker, Inc. 1999) can be used for guidance. All references mentioned herein are incorporated in their entirety.

### EXAMPLES

### X-Ray Powder Diffraction:

The x-ray powder diffraction was performed on a Scintag X-ray powder diffractometer model X'TRA with a solid state detector. Copper radiation of 1.5418 Å was used. The sample holder was a round standard aluminum sample holder with rough zero background. The scanning parameters were range: 2-40 degrees two-theta; scan mode: continuous scan; step size: 0.05 degrees; and at a rate of 3 degrees/minute. For peak calibration purposes, silica was mixed with the analyzed samples of crystalline ibandronate disodium Forms DS2 and DS3. The peak at about 28.5° 2θ±0.2° 2θ is due to the silica and not due to the sample of crystalline ibandronate disodium.

### Example 1

### Process for Preparing Amorphous Ibandronate Disodium

Ibandronic acid (5 g) was dissolved in water (33.4 ml, 6.67 vol.) at room temperature. NaOH(s) (1.25 g, 2 eq) was added to the solution. The solution was heated to reflux. Acetone (50 ml) was added drop-wise to the solution over 5 minutes. The solution was stirred at reflux for 10 minutes and then cooled to room temperature. The solution was stirred at room temperature for 2.5 hours to obtain a slurry. Acetone (50 ml) was added to the obtained slurry. The slurry was then stirred for 18 hours. The precipitate was isolated from the slurry by vacuum filtration under nitrogen, washed with acetone (1×15 ml) and dried in a vacuum oven at 50° C. for 19.5 hours to give 5.6 g of amorphous ibandronate disodium.

### Example 2

### Process for Preparing Crystalline Ibandronate Disodium Form DS2

Ibandronic acid (50 g) was dissolved in water (333.5 ml, 6.67 vol.) at room temperature. The solution was heated to reflux (70° C.). NaOH(s) (12.53 g, 2 eq) was added to the solution. Acetone (500 ml) was added drop-wise to the solution over 7 minutes at 92° C. The solution was stirred at reflux for 12 minutes and then cooled to room temperature. The solution was then stirred at room temperature for 80.5 hours to form a slurry. The precipitated was isolated from the slurry by vacuum filtration under nitrogen, washed with acetone (2×50 ml) and dried in a vacuum oven at 50° C. for 22.5 hours to give 45 g of ibandronate disodium Form DS2.

### Example 3

### Process for Preparing Crystalline Ibandronate Disodium Form DS3

300 mg amorphous ibandronate disodium was placed in a glass container at room temperature and the container placed in a chamber having 80% relative humidity. After one week of storage at room temperature and 80% relative humidity, the sample was analyzed by PXRD and surprisingly crystalline ibandronate disodium Form DS3 was obtained.

### Example 4

### Process for the Preparation of Crystalline Ibandronate Disodium Form DS4

300 mg amorphous ibandronate disodium was placed in glass container at room temperature and the container placed in a chamber having 60% relative humidity. After one weak of storage at room temperature and 60% RH the sample was analyzed by PXRD and form DS4 was obtained.

### Example 5

### Process for the Preparation of Crystalline Ibandronate Disodium Form DS3

300 mg amorphous ibandronate disodium was placed in glass container at room temperature and the container placed in a chamber having 100% relative humidity. After one weak of storage at room temperature and 100% RH the sample was analyzed by PXRD and form DS3 was obtained.

What is claimed is:

1. Ibandronate disodium.

2. Ibandronate disodium of claim 1 in solid form.

3. Ibandronate disodium of claim 2 in crystalline form.

4. Ibandronate disodium of claim 3 in hydrate form

5. Ibandronate disodium of claim 2 in amorphous form.

6. Ibandronate disodium of claim 5 characterized by an x-ray powder diffraction (“PXRD”) pattern as depicted in FIG. 1.

7. Ibandronate disodium of claim 5, having less than about 50% crystalline material as measured by area percentage XRD.

8. Ibandronate disodium of claim 7, having less than about 10% crystalline material.

9. Ibandronate disodium of claim 8, having less than about 1% crystalline material.

10. A process for preparing the amorphous form of claim 5 comprising combining ibandronic acid, water, a base and a source of sodium to obtain a solution, and combining the solution with acetone to precipitate amorphous form.

11. The process of claim 10, wherein amorphous ibandronate disodium is prepared by a process comprising dissolving ibandronic acid in water; adding a base and a source of sodium ions to the solution; heating the solution; adding acetone to the solution obtain a slurry; and cooling the slurry to precipitate amorphous ibandronate disodium.

12. The process of claim 10, wherein the ibandronic acid is dissolved in water at a temperature of about 15° C. to about 35° C. about room temperature.

13. The process of claim 10, wherein the base and the source of sodium ions is NaOH or Na₂CO₃.

14. The process of claim 11, wherein the solution is heated to about a temperature of about 90° C. to about 100° C. reflux temperature.

15. The process of claim 11, wherein the resulting slurry after addition of acetone is cooled to a temperature of about 15° C. to about 35° C. about room temperature to precipitate the amorphous ibandronate disodium.

16. The process of claim 10, further comprising recovering the amorphous form.

17. The crystalline ibandronate disodium of claim 3, wherein the crystalline form is characterized by PXRD reflections at 4.2, 12.8, 17.6, 19.9, and 20.3° 2θ±0.2° 2θ.

18. The crystalline ibandronate disodium of claim 17, further characterized by PXRD reflections at 12.4, 13.9, 17.0, 22.0, and 25.0±0.2° 2θ.

19. The crystalline ibandronate disodium of claim 18, further characterized by a PXRD pattern as depicted in FIG. 2.

20. The crystalline ibandronate disodium of claim 3, wherein the crystalline form is characterized by PXRD reflections at about 4.2, 12.8 and 17.6° 2θ±0.2° 2θ, and two more peaks selected from the group consisting of: 12.4, 13.9, 16.0, 17.0, 19.9, 20.3, 20.8, 22.0, 22.7, and 25.0±0.2° 2θ.

21. The crystalline ibandronate disodium of claim 20, characterized by a PXRD reflections at about 4.2, 12.4, 12.8, 16.0 and 17.6° 2θ±0.2° 2θ.

22. The crystalline ibandronate disodium of claim 3, wherein the crystalline form is characterized by PXRD reflections at about 12.4, 12.8 and 17.6° 2θ±0.2° 2θ, and two more peaks selected from the group consisting of: 4.2, 13.9, 16.0, 17.0, 19.9, 20.3, and 20.8±0.2° 2θ.

23. The crystalline ibandronate disodium of claim 22, characterized by a PXRD reflections at about 12.4, 12.8, 17.0, 17.6 and 19.9° 2θ±0.2° 2θ.

24. A process for preparing the crystalline form of claim 17, comprising combining ibandronic acid, water, a base and a source of sodium to obtain a solution, and combining the solution with acetone to obtain a slurry (heterogeneous mixture), and maintaining the slurry to precipitate crystalline ibandronate disodium.

25. The process of claim 24, wherein the process comprises dissolving ibandronic acid in water; heating the solution; adding a base and a source of sodium ions to the solution; heating the solution; adding acetone to obtain a slurry; and maintaining and cooling the slurry to precipitate the crystalline ibandronate disodium.

26. The process of claim 24, wherein the crystalline form is obtained via amorphous form as an intermediate.

27. The process of claim 24, wherein the slurry is maintained for a period of more than about 20 hours.

28. The process of claim 27, wherein the slurry is maintained for more than about 40 hours.

29. The process of claim 25, wherein ibandronic acid is dissolved in water at a temperature of about 15° C. to about 35° C. about room temperature.

30. The process of claim 24, the base and the source of sodium ions is NaOH or Na₂CO₃.

31. The process of claim 25, the solution is heated to a temperature of about 90° C. to about 100° C. to about reflux temperature.

32. The process of claim 24, wherein the resulting slurry is cooled to a temperature of about 10° C. to about 35° C., to about room temperature to precipitate the crystalline ibandronate disodium.

33. The process of claim 24, wherein the crystalline ibandronate disodium is recovered by collecting the precipitate from the slurry by filtration.

34. The process of claim 24, wherein the crystalline ibandronate diNa having a maximal particle size of less than about 500 μm.

35. The crystalline ibandronate disodium form of claim 3, wherein the crystalline form is characterized by PXRD reflections at 4.6, 9.5, 14.9, and 17.3° 2θ±0.2° 2θ.

36. The crystalline form of claim 35, further characterized by PXRD reflections at 9.0, 11.9,21.5,23.7, and 27.5° 2θ±0.2° 2θ.

37. The crystalline ibandronate disodium of claim 36, further characterized by a PXRD pattern as depicted in FIG. 3.

38. The crystalline ibandronate disodium form of claim 3, wherein the crystalline form is characterized by PXRD reflections at 4.6, 9.5 and 14.9° 2θ±0.2° 2θ, and two more peaks selected from the group consisting of: 9.0, 11.9, 16.0, 16.7, 17.3, 18.1, 21.0, and 22.5±0.2° 2θ.

39. The crystalline ibandronate disodium form of claim 38, characterized by PXRD reflections at 4.6, 9.0, 9.5, 14.9 and 17.3° 2θ±0.2° 2θ.

40. The crystalline ibandronate disodium form of claim 3, wherein the crystalline form is characterized by PXRD reflections at 9.5, 14.9 and 17.3° 2θ±0.2° 2θ, and two more peaks selected from the group consisting of: 9.0, 11.9, 16.0, 16.7, 18.1, 21.0, and 22.5±0.2° 2θ.

41. The crystalline ibandronate disodium form of claim 40, characterized by PXRD reflections at 9.0, 9.5, 14.9, 16.0 and 17.3° 2θ±0.2° 2θ.

42. A process for preparing the crystalline ibandronate disodium of any of claim 35, comprising contacting ibandronate disodium with water vapors at a relative humidity of above about 80%.

43. The process of claim 42, wherein the process comprises storing amorphous ibandronate disodium at a temperature of about 10° C. to about 30° C., at about 80% to about 100% relative humidity, for about 2 days to about 7 days, preferably about 7 days.

44. The process of claim 43, wherein the amorphous ibandronate disodium is stored at about room temperature.

45. The process of claim 43, wherein amorphous ibandronate disodium is stored at about 80% relative humidity.

46. The crystalline ibandronate disodium of claim 35, having a maximal particle size of less than about 500 μm.

47. The crystalline ibandronate disodium form of claim 3, wherein the crystalline form is characterized by PXRD reflections at 3.7, 10.9 and 14.4° 2θ±0.2° 2θ, and two more peaks selected from the group consisting of: 7.3, 13.6, 15.9, 20.7±0.2° 2θ.

48. The crystalline ibandronate disodium form of claim 47, characterized by PXRD reflections at 3.7, 7.3, 10.9, 13.6 and 14.4° 2θ±0.2° 2θ.

49. The crystalline ibandronate disodium form of claim 3, wherein the crystalline form is characterized by PXRD reflections at 10.9, 13.6 and 14.4° 2θ±0.2° 2θ, and two more peaks selected from the group consisting of: 7.3, 13.6, 15.9, 20.7, 22.6, and 27.4±0.2° 2θ.

50. The crystalline ibandronate disodium form of claim 49, characterized by PXRD reflections at 7.3, 10.9, 13.6, 14.4 and 15.9° 2θ±0.2° 2θ.

51. A process for preparing the crystalline ibandronate disodium of claim 49, comprising contacting ibandronate disodium with water vapors at a reltive humidity of less than about 75%.

52. The process of claim 50, wherein the process comprises storing amorphous ibandronate disodium at a temperature of about 10° C. to about 30° C., at about 60% relative humidity, for about 2 days to about 7 days, preferably about 7 days.

53. The process of claim 51, wherein the amorphous ibandronate disodium is stored at about room temperature.

54. A pharmaceutical composition comprising one of amorphous form or crystalline form of ibandronate disodium and at least one pharmaceutically acceptable excipient.

55. The pharmaceutical composition of claim 54, wherein the crystalline form is Form DS2.

56. The pharmaceutical composition of claim 54, wherein the crystalline form is Form DS3.

57. A process for preparing ibandronate disodium comprising maintaining a mixture of amorphous ibandronate disodium in acetone and water.

58. A method of treating or preventing osteoporosis comprising administering the pharmaceutical composition of claim 54 to a subject.
